(19) United States
(12) Patent Application Publication
(10) Pub. No.: US 2014/0221968 A1
(43) Pub. Date: Aug. 7, 2014

(54) DEVICES AND METHODS FOR IMPROVED VASCULAR ACCESS

(71) Applicant: Nocturnal Product Development, Cary, NC (US)

(72) Inventors: Terry Ransbury, Chapel Hill, NC (US); Robert Smith, Raleigh, NC (US); Mark Urtz, Raleigh, NC (US)

(21) Appl. No.: 14/152,714

(22) Filed: Jan. 10, 2014

Related U.S. Application Data
(60) Provisional application No. 61/751,036, filed on Jan. 10, 2013.

(51) Int. Cl. A61M 39/06

(52) U.S. Cl. CPC A61M 39/06; A61M 2039/0626; A61M 2210/005
USPC 604/506; 604/256

(57) ABSTRACT

Devices and methods of use for obtaining and maintaining vascular access for the insertion of diagnostic or therapeutic drugs or devices into a patient. A device may include a first conduit extending along a longitudinal axis between a first end and a second end, the first end configured for coupling to a needle and the second end configured for coupling to a syringe. The device also includes a second conduit oblique to the first conduit and in communication with the first conduit. And the device includes a valve in communication with the second conduit, the valve being operatable between a closed position and an open position, wherein the open position is configured to provide access to the second conduit, such that an object may be introduced through the valve and second conduit into the first conduit and out the needle.

### CROSS REFERENCE TO RELATED APPLICATIONS

The present invention claims the benefit of U.S. Provisional Application No. 61/751,036, filed Jan. 10, 2013, the disclosure of which is hereby incorporated by reference in its entirety.

### FIELD OF THE INVENTION

The present invention generally relates to vascular medical procedures. In particular, the invention provides methods and systems for improved success rates and faster procedures that require obtaining and maintaining vascular access for the insertion of diagnostic or therapeutic drugs or devices.

### BACKGROUND OF THE INVENTION

Obtaining and maintaining vascular access, be it arterial or venous, is common to many medical procedures where the introduction of an introducer sheath or central line is the first step towards diagnosis and treatment of the patient. Once the sheath or central line is in place, the physician has vascular access with which drugs or medical devices can be delivered anywhere along the vascular system. A few examples of such medical procedures that involve the insertion of devices are: cardiac catheter procedures, peripheral vascular procedures, neurological vascular procedures, and cardiac pacemaker or defibrillator lead implantation. Drug delivery, including anesthesia, also begins with proper vascular access.

The traditional means and methods of obtaining and maintaining vascular access are well known within the medical community as variants of the Modified Seldinger technique. This technique can be used anywhere vascular access is needed but the most common sites are the neck for jugular access, the groin for femoral access, or the arm for brachial access.

The tools currently used in the Modified Seldinger technique are a hypodermic needle, a syringe, sterile saline fluid, an optional guide wire, and an optional sheath or central line.

The needles come in many sizes of length and diameter depending upon the needs of the procedure. The syringes also come in many sizes that are more or less convenient to the type and amount of fluid that needs to be collected or dispensed. The needle and syringe can be mated and unmated though a threaded connector set that has become a generally accepted standard. The connector set (male and female) is called a Luer connector. Typically, the Luer connector on the needle is “female” while the connector on the syringe is “male.” Also typically, the female connector is fixed while the male connector may be either fixed or rotatable for connection.

The wire, while not necessary in all vascular access procedures, is inserted into the needle once the syringe is removed. The wire allows the needle to be removed while maintaining access to the vessel. This allows the insertion of the sheath or central line over the wire and into the vessel.

The sheath or central line is the final tool used in the Modified Seldinger technique. It maintains access to the vessel for the specific devices and drugs to be administered. It also provides hemostasis (prevention of blood loss) over time. When vascular access is no longer needed, the sheath is removed and discarded.

The above tools are used in the currently practiced Modified Seldinger technique procedure, wherein an anatomical site of choice is prepared and sterilized, the needle is attached to the syringe and a small amount of sterile saline fluid is pulled into the syringe. The physician ejects a bit of the saline to displace any air that may be in the needle or syringe and thus prevents the introduction of air into the bloodstream that can be harmful to the patient. The physician then probes the skin for a puncture site with the hypodermic needle. As the physician punctures the skin and is searching for the target vein or artery, he simultaneously pulls back on the plunger of the syringe drawing blood into the needle and syringe. The trained and experienced physician looks at characteristics of the blood (color, velocity of entering the syringe, etc.) in the syringe to determine if the needle is in the appropriate vein or artery. If unsure, the syringe is removed from the needle and the blood chemically tested to determine its source.

If the blood is from the wrong vessel, the probing is repeated until the successful vessel is found or the syringe is full. If the syringe is full, the needle is removed from the site and the blood expelled from syringe and the searching is repeated.

Once the target vessel has been accessed by the needle and the proper blood type is seen, the physician removes the syringe from the needle being careful not to displace the needle tip out of the target vessel. In the case of a fixed Luer connector, the physician twists the syringe from the needle to remove it. In the case of, the rotating Luer connector, the physician unscrews the connector to remove the syringe.

The removal of the syringe enables the exposed end of the hollow needle to accept the guide wire. The physician feeds the guidewire through the opening of the needle and then pulls the needle out of the patient over the wire, leaving it in place for the insertion of an introducer sheath or central line for indefinite vascular access.

In the Modified Seldinger technique, described above, once the needle tip is in place within the intended blood vessel, the syringe must be removed without displacing the needle. In the cases of procedures with device intervention, the syringe must be removed so that the guidewire can be inserted into the needle to maintain vessel access for the subsequent removal of the needle and placement of an introducer sheath. In the cases of drug intervention, the syringe must be removed so that a different syringe can be connected to the needle to administer the drug.

Common to both types of interventions, the jarring nature of the disconnection of the syringe as it is twisted from the needle often, and accidentally, displaces the tip of the needle from the intended target vessel. The needle tip then ends up located in either non-vessel tissue or perhaps an unintended artery or vein, since the two often run in close proximity. Moreover, once the syringe is removed, there is no indication that the needle has moved because the blood sample in the syringe no longer reliably indicates correct or current position. If displaced, the physician must start over from the beginning of the vascular access procedure. It is not hard to imagine that consequences of such error can be more than just increased physician frustration and increased procedure time.

FIG. 1A includes the two components to form the majority of prior art when it comes to vascular access: a standard syringe 100 and a mating hypodermic needle 200. The two components are mated and unmated by a standard connector type familiar in the medical community called a Luer connection 101. Although arbitrary, the typical arrangement is shown where the male Luer 102 belongs to the syringe and the female Luer 103 to the needle. There are two variants of the male Luer 102 commonly available on the syringe 100, one with a fixed connector orientation and the other a rotating connector. Each of the variants looks similar to the connector in the figure. Either configuration can be used in the standard approach to vascular access and the proposed improved approaches. For simplicity, the guide wire and introducer sheath or central line are omitted but well understood to those in the art.

FIGS. 1B-E are plan views showing prior art of a single-handed syringe 120, as described in U.S. Pat. No. 4,484,915 to Tartaglia. FIG. 1B shows the single handed syringe 120 in the position where all fluid 410 is expelled from the syringe 120. The arrow in FIG. 1C shows the points of contact on the single handed syringe 120 that are moved to cause the syringe 120 to take in fluid 410. Specifically, although acknowledged relative, point 110 is held stationary and point 112 is moved towards 110 to move the plunger 105 to the left in the figure. This works because the ring 107 is coupled mechanically to the plunger 105 by struts 106. The struts 106 are shown at 90 degree angles in FIGS. 1C and 1D. In the reverse and in order to expel fluid 410 from the syringe 120, ring 107 is held stationary and the physician moves the plunger 105 to the right in the diagram. This is the same technique used for standard syringes 100.

FIGS. 2A-D show the sequence of the current Modified Seldinger Technique and illustrate the problem that results anatomically when the needle tip 202 is displaced during syringe 100 removal. FIG. 2A shows the standard syringe 100 inserted through the skin layer 402, through non-vessel tissue 403, and into the target vessel 404. FIG. 2B shows the next two steps in the vessel access procedure. First, the standard syringe 100 withdraws blood 411 from the vessel as the physician 420 pulls on the plunger 105 away from the syringe body 104. The second step shows a rotation 160 of the syringe 100 to disconnect it from the needle 200. If done successfully, the needle tip 202 remains in the target vessel 404. However, as shown in FIG. 2C, if the needle 200 is displaced during the removal of the syringe 100, the needle tip 202 can end up outside the blood vessel 404 and into non-vessel tissue 403. Likewise, as shown in FIG. 1D, a displaced needle 200 can end up in an unintended blood vessel 408.

FIGS. 3A-E show the successful sequence of the Modified Seldinger Technique as is the state of the art today. Here, the standard syringe 100 and needle 200 system are shown mated in FIG. 3A with a small amount of saline 412 in the syringe 100 to purge the air 414. Once inserted through the skin layer 402 and into the target blood vessel 404, the plunger 105 is retracted to draw blood 411 into the syringe 100. The physician examines the blood 411 to determine whether or not the needle 200 is in the target vessel 404 (vein or artery). If in the correct vessel type, the syringe 100 is removed and the needle 200 remains as shown in FIG. 3C. At this point, the vessel will bleed through the exposed end of the needle 200 unless the physician 420 restricts the bleeding by, for example, holding a thumb over the opening. FIG. 3D shows the guide wire 204 inserted through the needle 200 and into the target blood vessel 404. Next the physician 420 removes the needle 200 leaving the guide wire 204 in place to maintain the vascular access shown in FIG. 3E. With suitable vascular access established, an introducer sheath or central line can then be inserted over the guide wire.

A need exists for improved devices and methods for obtaining and maintaining access to the vasculature of a patient.

### SUMMARY OF THE INVENTION

The present application describes a system and method to improve vascular access for physicians. The system and method improve the stability of the needle under the skin and in the target blood vessel. This improves the success rate of vascular access and consequently reduces the time of procedures. It may also improve the safety of the procedure for the patient. The described system builds upon the tools widely in use today for vascular access including: a syringe, hypodermic needle, guidewire, and introducer sheath (or central line). The described system enhances the vascular access procedure in two ways. First, by adding a component called the inline wire port (IWP), and second, by an improved syringe that is optimized to the vascular access procedure.

The key functions of the IWP are: maintaining hemostasis (i.e. not allowing blood to leak out of the system), maintaining an air-tight seal (i.e. not allowing air to be introduced into the system and therefore patient), the facilitation of guide wire placement without the need to disconnect the syringe, easier syringe manipulation with one hand, and maintaining the current ergonomics of a straight in-line arrangement of the syringe and needle that the physicians are accustomed.

The present invention also describes an improved syringe that is optimized for the particular procedure of vascular access. It does so through better ergonomics of the syringe allowing for smoother, single-handed operation and thus more stability of the needle tip while the blood sample, indicating the needle tip position, is drawn.

Lastly, the present invention describes a system that maintains the in-line orientation of the syringe and the needle as used today. This orientation is important to the physicians because, that is the way they were trained and it maximizes the accuracy of needle placement.

### BRIEF DESCRIPTION OF THE DRAWINGS

The invention may be more completely understood in consideration of the following detailed description of various embodiments of the invention in connection with the accompanying drawings, in which:

FIG. 1A shows components used for vascular access in the current art.

FIG. 1B is a plan view showing prior art of a single-handed syringe.

FIG. 1C is a plan view showing how the syringe of FIG. 1B takes in fluid.

FIG. 1D is a plan view showing the syringe of FIG. 1B rotated 90 degrees.

FIG. 1E is a plan view showing how the syringe of FIG. 1B expels fluid.

FIG. 2A is a plan view showing as standard syringe with the needle inside a vessel.

FIG. 2B is a plan view showing how a standard syringe withdraws blood from the vessel.

FIG. 2C is a plan view showing how the removal of the syringe of FIG. 2B can leave the needle outside a vessel.

FIG. 2D is a plan view showing how the removal of the syringe of FIG. 2B can leave the needle inside an unintended vessel.

FIG. 3A is a plan view showing the starting point of the vascular access procedure with the components representing the current art.

FIG. 3B is a plan view showing the syringe of FIG. 3A activated to draw blood.

FIG. 3C is a plan view showing the needle after the syringe has been removed from FIG. 3B.

FIG. 3D is a plan view showing the guide wire inserted through the needle of the progression commencing with FIG. 3A.

FIG. 3E is a plan view showing the resultant guide wire of the progression commencing with FIG. 3A.

FIG. 4A is a plan view showing a first embodiment with the invention as a discrete component of the system and a variant of an ergonomically improved syringe.

FIG. 4B is a plan view showing a second embodiment of the system with another variant of ergonomically improved syringe.

FIG. 4C is a plan view showing a third embodiment of the system with the primary invention integrated with the hypodermic needle.

FIG. 4D is a plan view showing a fourth embodiment of the system with the primary invention integrated with both the syringe and the hypodermic needle.

FIG. 5A is a plan view showing the starting point of the vascular access procedure with a standard syringe and needle with an embodiment of the invention.

FIG. 5B is a plan view showing the syringe of FIG. 5A activated to draw blood.

FIG. 5C is a plan view showing the needle after the syringe has been removed from FIG. 5B.

FIG. 5D is a plan view showing the guide wire inserted through the needle of the progression commencing with FIG. 5A.

FIG. 5E is a plan view showing the resultant guide wire of the progression commencing with FIG. 5A.

FIG. 6 is a plan view showing the starting point of the vascular access procedure with a standard needle with an embodiment of an ergonomically improved syringe and the invention.

FIG. 7 is a plan view showing an alternate embodiment of the invention where all of the components are integrated.

FIG. 8 is an alternate embodiment of the system.

FIG. 9A is a plan view of a variant of the invention component only.

FIG. 9B is an enlarged cross-sectional view of a subsection of FIG. 9A.

FIG. 9C shows cross-sectional view and an end view of a subsection of FIG. 9B.

FIG. 9D is an enlarged cross-sectional view of a subsection of FIG. 9A with the valve function in the open position.

FIG. 9E is an end view of FIG. 9D with the valve function in the open position.

FIG. 10A is a plan view of a variant of the invention component only.

FIG. 10B is an enlarged cross-sectional view of a subsection of FIG. 10A.

FIG. 10C shows cross-sectional view and an end view of a subsection of FIG. 10B.

FIG. 10D is an enlarged cross-sectional view of a subsection of FIG. 1 OA with the valve function in the open position.

FIG. 10E is an end view of FIG. 10D with the valve function in the open position.

FIG. 11A is a plan view of a variant of the invention's valve component only.

FIG. 11B is an enlarged cross-sectional view of a subsection of FIG. 11A with the valve function in the closed position.

FIG. 11C is an enlarged cross-sectional view of a subsection of FIG. 11A with the valve function in the open position.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

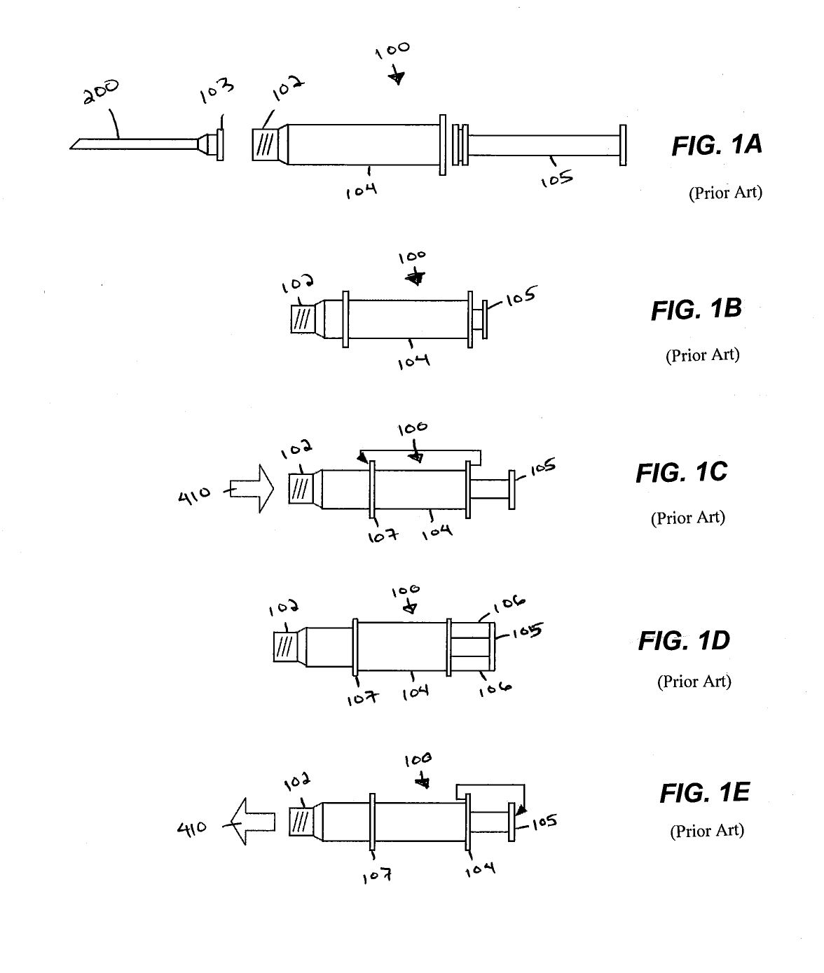

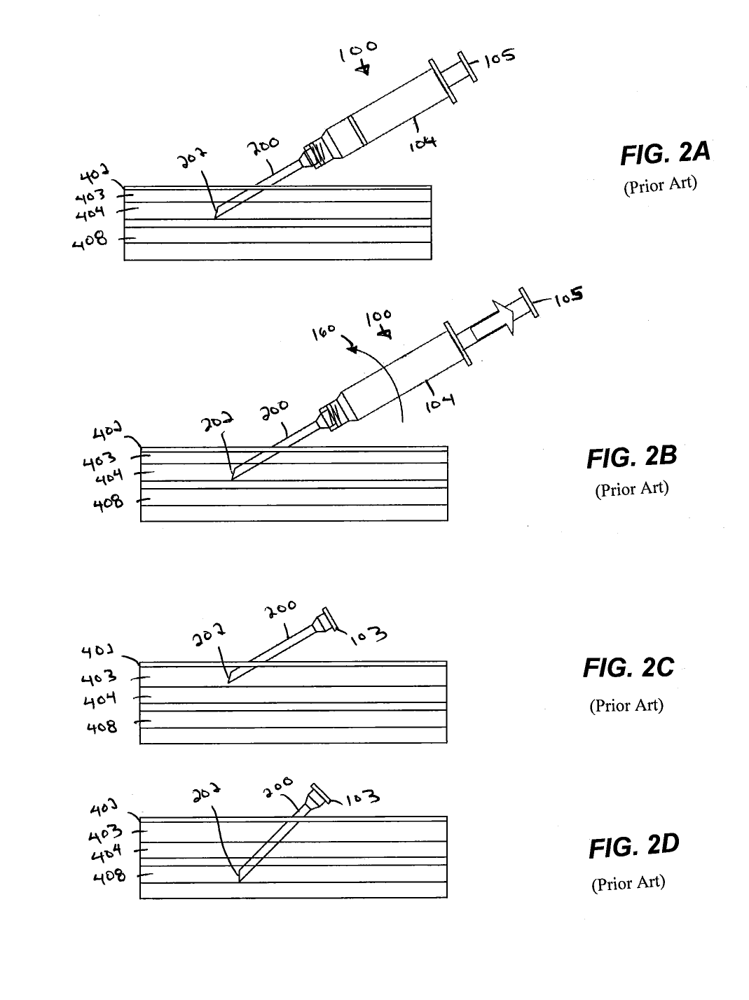

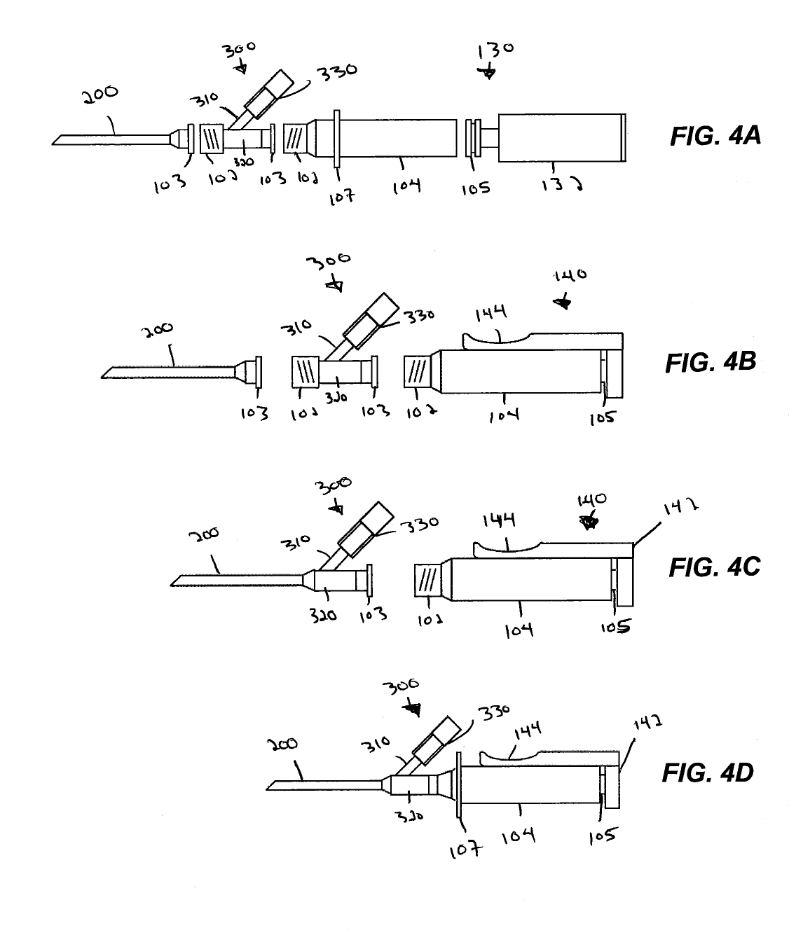

FIGS. 4A-4D show various embodiments with improvements to the standard needle and syringe configuration shown in FIG. 1A. In one embodiment, FIG. 4A shows two such improvements. The first is the addition of the Inline Wire Port (IWP) 300. The second improvement one embodiment of a single-handed syringe 130. In some embodiments, the IWP 300 comprises a valve stem 310, longitudinal shaft 320, pinch valve 330, and wire entrance 304. In some embodiments, IWP 300 comprises a male Luer connector 102 for the syringe 130 and a female Luer connector 103 for the needle 200 so that mating to a variety of syringes and needles are maintained. In FIG. 4A, the modified syringe 130 comprises a plunger 105 and an outer member 132 operably connected so that the outer member 130 directly manipulates the plunger 105 of the modified syringe 130. This allows the physician 420 to withdraw fluid 410 into the modified syringe 130 with a single hand while not requiring a repositioning of the hand back to the plunger 105.

In another embodiment, FIG. 4B shows an ergonomic single handed syringe 140. The ergonomic single handed syringe 140 comprises a plunger 105 operably connected to an outer member 142, wherein the outer member 142 comprises a thumb groove 144. The thumb groove 144 is ergonomically shaped to better fit the physician's hand and thumb, thus providing better traction than embodiments without such a thumb groove 144. The movement of the single handed ergonomic syringe 140 is easier on the hand for drawing blood 411 during the procedure and provides more stability to the tip 202 of the needle 200 than a standard syringe 100.

In another embodiment, FIG. 4C shows single partially integrated kit 500 comprising an IWP 300 and needle 200. This embodiment may be preferred over the embodiments of FIGS. 4A and 4B for cost reasons, as it is a simpler part, and for its ability to guarantee the special relationship between the beveled tip 202 of the needle 200 (which the physicians 420 use consistently as shown) and the inline wire port (IWP) 300. This relationship allows the physician to use one hand (usually the left) to hold the needle 200 in place, while the other (usually right) can release from the syringe 140 and manipulate the wire 302 and wire port 304. This assembly can be made with many different sizes (gauges and lengths) of needles 200 that would work seamlessly in the new methods described later in the text.

In another embodiment, FIG. 4D shows a fully integrated kit 600 comprising yet an IWP 300, needle 200, and an ergonomic single handed syringe 140. In some embodiments, the fully integrated kit 600 includes a leverage ring 107 that serves as a leverage point for the index finger countering the force of the thumb to slide the plunger 105 back. This embodiment and the one in FIG. 4C may be preferred over the embodiments of FIGS. 4A and 4B for cost reasons, as they comprise simpler parts, and yet they carry all the benefits of the other embodiments.

It is important to note the subtle, but critical, difference between the prior art of FIGS. 1A-E and the proposed embodiments of the syringe in FIGS. 4A-D. In the proposed invention, these syringes can be manipulated without repositioning the hand like is required between the different states of the prior art. The reduction of hand movement in the proposed embodiments contributes to the overall stability of the system and hence the desired stability of the needle tip 202 within the target blood vessel 404.

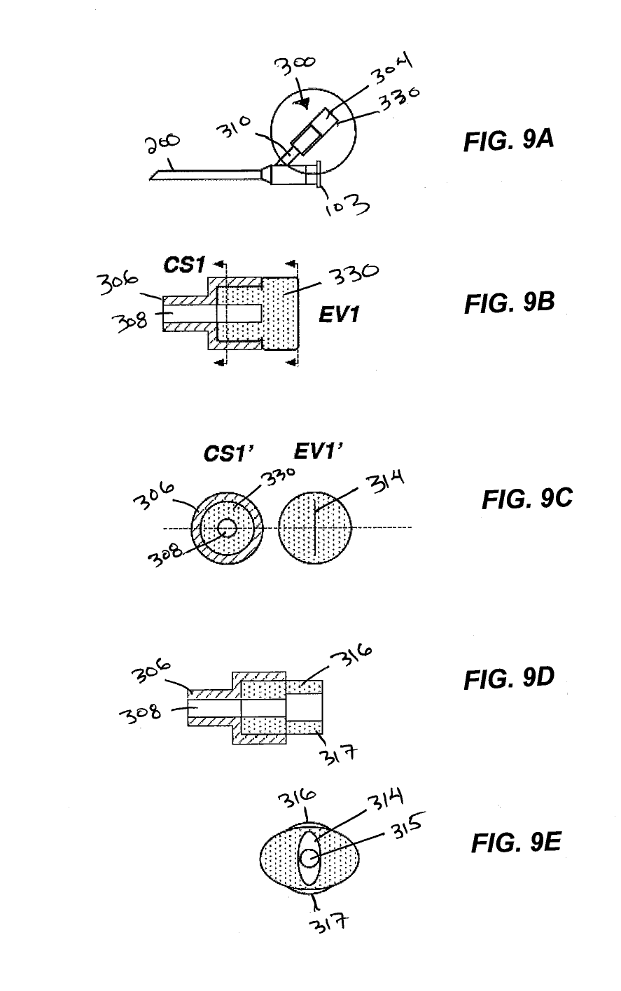

This embodiment of the valve function of the IWP 300 can be instantiated in many ways familiar to those versed in the art without compromising the intent of the invention. One example would be for the valve to be a Tuhoy-Borst valve which is well known in the community. There are many variants that use either a push method or a screw method to open and close the valve. However, each of these techniques are less than ideal choices when compared to the pinch valve described in FIG. 9. The actions of opening and closing the pinch-type valve is a motion perpendicular to the insertion of the needle and is therefore inherently more stable.

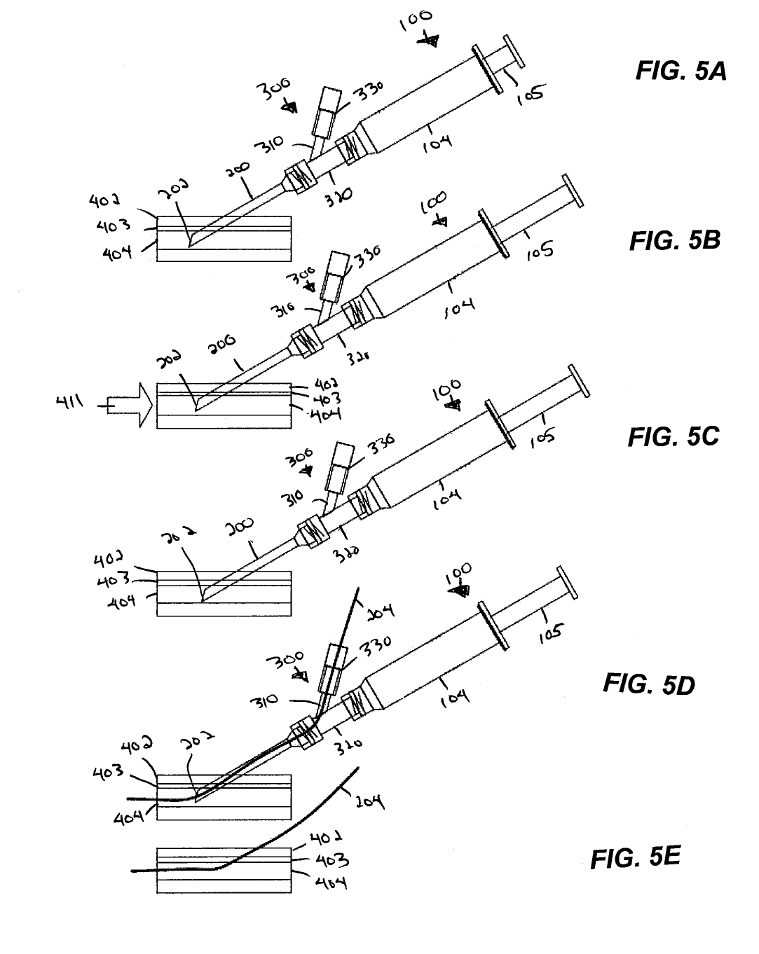

FIGS. 5A-5E show one embodiment of the proposed new vascular access system and method. In FIG. 5A, the standard syringe 100, one particular embodiment of the IWP invention 300 and a standard needle 200 are mated together and inserted through the skin layer 402 and into the target blood vessel 403. In FIG. 5B, the plunger 105 of the standard syringe 100 is retracted to draw blood 411 into the syringe 100. The physician 420 examines the blood 411 to determine whether or not the needle is in the target blood vessel 404 (vein or artery).

Here begins the difference in the proposed method over the current standard method. If the needle 200 is in the target blood vessel 404, the physician 420 does not need to remove the standard syringe 100. Therefore, the physician 420 avoids a disturbance of the needle 200 that may leave its tip 202 outside of the target vessel 404. In FIG. 5C., the physician 420 opens the wire pinch valve 330. In FIG. 5D, the physician 420 inserts a guide wire 204 through the wire port entrance 304 (the detail of the valve operation is described later in FIGS. 9A-E). The guide wire 204 continues through the pinch valve 300, valve stem 310, longitudinal shaft 320, needle 200, ending up in the target blood vessel 404.

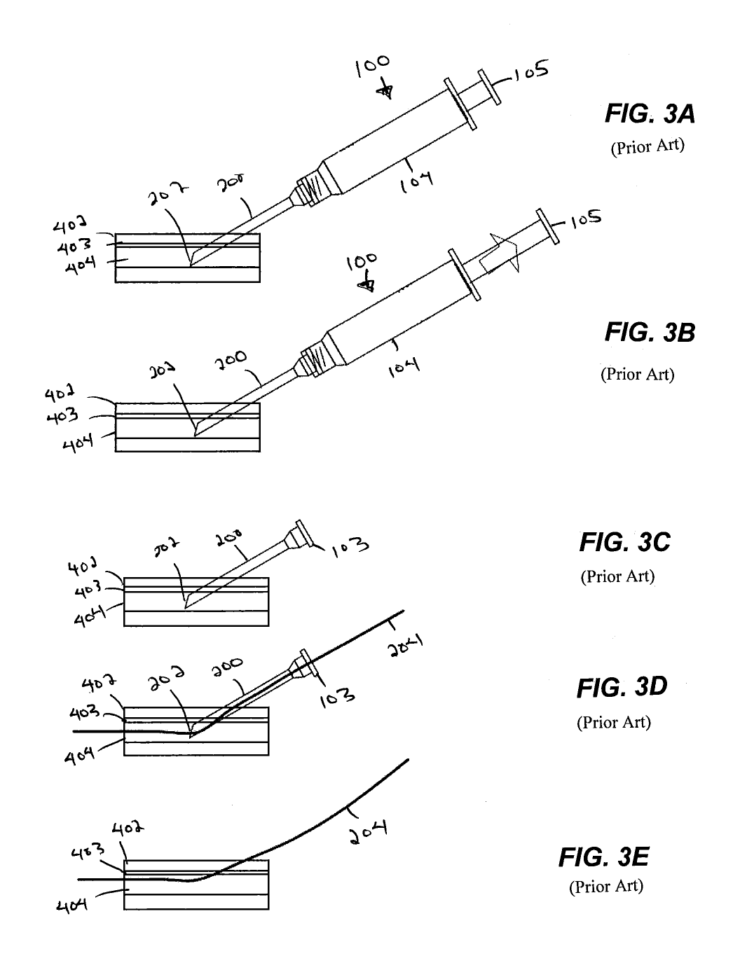

Next the physician 420 removes the entire system leaving the guide wire 204 in place to maintain the vascular access shown in FIG. 5E. Again, not shown for brevity, are the remaining steps of inserting the introducer sheath or central line over the guide wire and then removing the guide wire. This leaves the sheath to serve as the access vehicle for subsequent physician intervention. It should be noted also that in contrast to the standard procedure shown in FIGS. 3A-3E, the IWP 300 can minimize blood loss due to its sealable wire port valve.

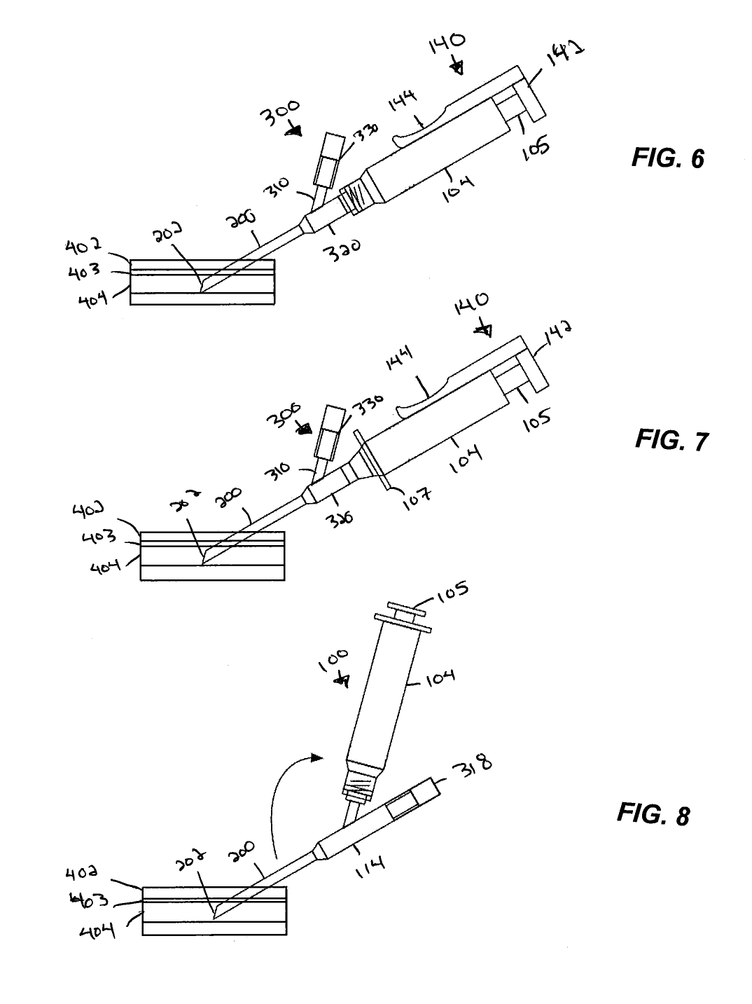

FIG. 6 shows the same new method as described in FIGS. 5A-E, except for two important differences. First, the standard syringe 100 is replaced with the single handed ergonomic syringe 140. Second, physician 420 uses the partially integrated kit 500 described above. Both of these improvements would make this system configuration the preferred embodiment.

FIG. 7 shows the same method as described in FIG. 6; however, the physician 420 uses the fully integrated kit 600 as described above.

FIG. 8 shows an alternate embodiment of the system. Here, a standard syringe 100 and a standard needle 200 can be mated to a similar Y-type connector 114 which is similar to IWP 300 described above. The standard syringe 100 and needle 200 are oriented at an angle of about 135 degrees.

FIGS. 9A-E show details of one example of many techniques to provide a simple valve to control the wire access and provide hemostasis.

Referring to FIG. 9A, one embodiment of the system comprises a partially integrated kit 500 as described above. In some embodiments, the IWP 300 of the partially integrated kit 500 comprises a valve stem 310, longitudinal shaft 320, pinch valve 330, and wire entrance 304. FIG. 9B shows a cross sectional view of the detail in FIG. 9A. In some embodiments, the valve stem 310 comprises a rigid housing 306 and hollow section 308. The hollow section 308 serves as the conduit for the wire while also supporting the pinch valve 330. The rigid housing 306 can be made of many biocompatible materials known to those familiar in the art including many silicones, plastics, and polyurethanes. The pinch valve 330 can be a simple molded silicone part with a continuation of the wire conduit and a valve slit 314 that opens and closes. The pinch valve 330 can be glued in place or trapped by a cap (not shown) that is glued or threaded to the housing 306.

FIG. 9C shows the cross section (CS1) and end view (EV1) of the valve in the closed position 324.

FIGS. 9D AND 9E show the pinch valve 330 in operation. Referring to FIG. 9D, the pinch valve 330 is opened when the physician 420 pinches the sides 316 and 317, which are approximately directly across from each other. Referring to FIG. 9E, when the pinch valve 330 is pinched, the compression of the slit 314 reveals the valve opening 315.

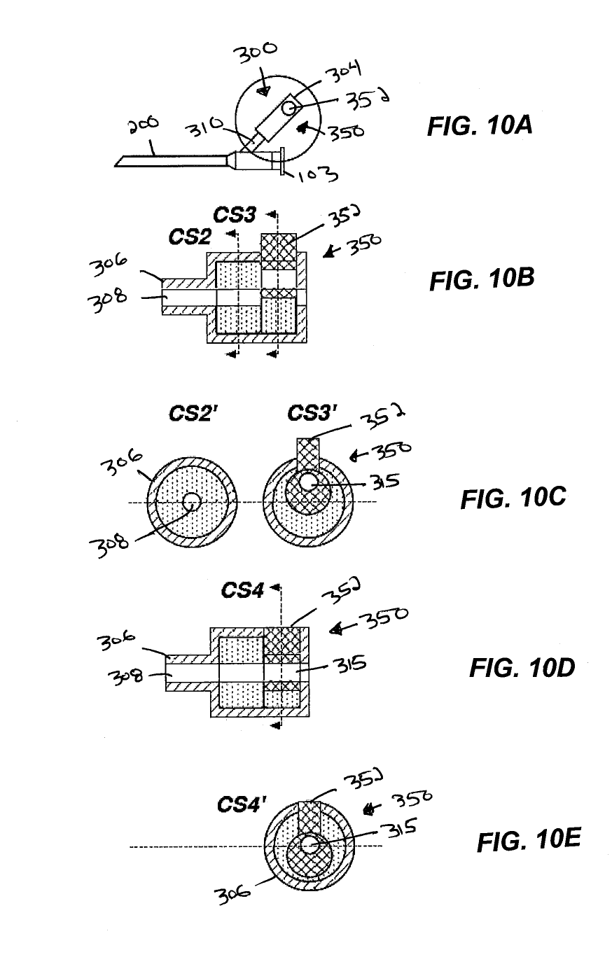

FIGS. 10A-E show another embodiment of the IWP 300 and the method of using a simple valve to control the wire access and provide hemostasis.

Referring to FIG. 10A, one embodiment of the system comprises a partially integrated kit 500 as described above. In one embodiment, the IWP 300 of the partially integrated kit 500 comprises a valve stem 310, longitudinal shaft 320, button valve 350, and wire entrance 304. FIG. 10B shows a cross sectional view of the detail in FIG. 10A. In some embodiments, the valve stem 310 comprises a rigid housing 306 and hollow section 308. The hollow section 308 serves as the conduit for the wire while also supporting the button valve 350. The rigid housing 306 can be made of many biocompatible materials known to those familiar in the art including many silicones, plastics, and polyurethanes. The button valve 350 can be a simple molded silicone part with a continuation of the wire conduit and a valve opening 315. The button valve 350 can be glued in place or trapped by a cap (not shown) that is glued or threaded to the housing 306.

FIG. 10C shows the cross section (CS1) and end view (EV1) of the valve in the closed position 324.

FIGS. 10D AND 10E show the button valve 350 in operation. Referring to FIG. 10D, the button valve 350 is opened when the physician 420 pushes on the button tab 352. Pressing the button 352 on the button valve 350 causes the valve opening 315 to align with the hollow section 308 of the valve stem 310. FIG. 10E, shows an end view of the opening that is created when the valve opening 315 is aligned with the hollow section 308.

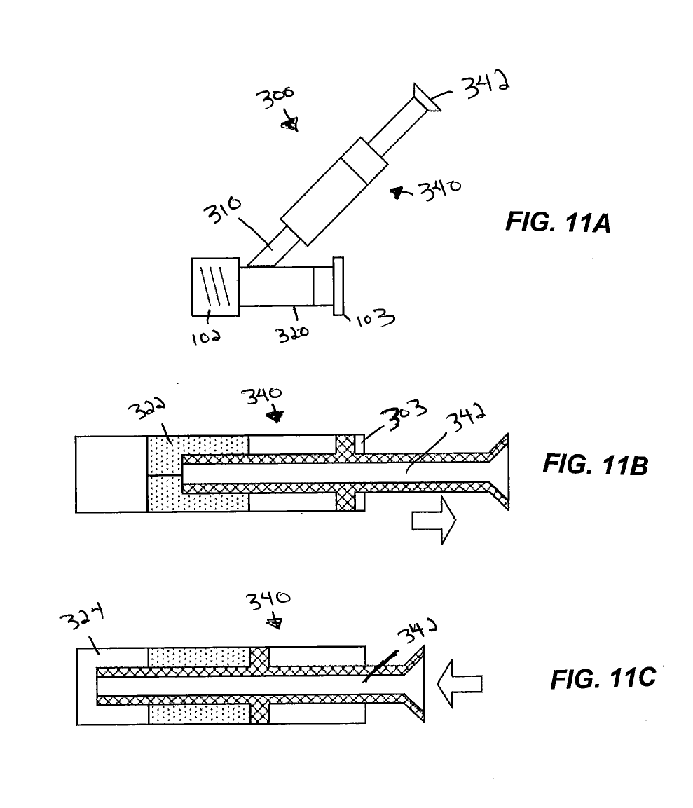

Referring to FIGS. 11A-C, one embodiment of the IWP 300 comprises a push valve 340 rather than a push valve 330 or button valve 350. The push valve 340 comprises a valve 312 and a wire port 342 having a conduit therein. FIG. 11B shows the valve in the closed position 322. As the wire port 342 is retracted (in this example to right in the figure) it does not penetrate the silicone valve 312. The wire port 342 has restricted travel within the valve by a flange 303 to facilitate open and closed positions but not allowing it to escape from the IWP 300. FIG. 11C is an enlarged cross-sectional view of a subsection of FIG. 11A with the valve function in the open position 324. Here, the wire port 342 is moved (in this example to the left extreme of travel in the figure) so that it penetrates beyond the silicone valve 312 allowing for the guide wire 204 (not shown here but in many other figures) to move through the conduit of push valve 340.

In another embodiment, instructions for using a vascular access device in accordance with the various embodiments described herein in the form of printed or electronically, optically or magnetically stored information to be displayed, for example, are provided as part of a kit or assemblage of items prior to usage of the vascular access device. The kit may be comprised of one or more hermetically sealed and sterilized packages, including the necessary components of the vascular access device as shown and described in the various embodiments herein.

Various modifications to the embodiments of the inventions may be apparent to one of skill in the art upon reading this disclosure. For example, persons of ordinary skill in the relevant art will recognize that the various features described for the different embodiments of the inventions can be suitably combined, un-combined, and re-combined with other features, alone, or in different combinations, within the spirit of the invention. Likewise, the various features described above should all be regarded as example embodiments, rather than limitations to the scope or spirit of the inventions. Therefore, the above is not contemplated to limit the scope of the present inventions.

Persons of ordinary skill in the relevant arts will recognize that the inventions may comprise fewer features than illustrated in any individual embodiment described above. The embodiments described herein are not meant to be an exhaustive presentation of the ways in which the various features of the inventions may be combined. Accordingly, the embodiments are not mutually exclusive combinations of features; rather, the inventions may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the embodiments of the present inventions, it is expressly intended that the provisions of Section 112, sixth paragraph of 35 U.S.C. are not to be invoked unless the specific terms “means for” or “step for” are recited in a claim.

1. A device for use in obtaining and/or maintaining access to a vasculature of a patient, the device comprising:
a first conduit extending along a longitudinal axis between a first end and a second end, wherein the first end is configured for coupling to a needle and the second end is configured for coupling to a syringe;
a second conduit, oblique to the first conduit and in communication with the first conduit; and
a valve in communication with the second conduit and configured to selectively seal an end of the second conduit, the valve being operatable between a closed position and an open position, wherein the open position is configured to provide access to the second conduit such that an object may be introduced through the valve and second conduit into the first conduit and out the needle.

2. The device of claim 1, wherein the valve is operatable between the closed position and the open position in a direction that is transverse to the longitudinal axis of the device.

3. The device of claim 1, wherein the valve is configured to be pinched to move from the closed position to the open position.

4. The device of claim 1, wherein the valve is operatable between the closed position and the open position in a direction that is parallel to the longitudinal axis of the device.

5. The device of claim 1, wherein the valve is biased to the closed position.

6. The device of claim 1, wherein the first end comprises a means for coupling to a needle, and wherein the second end comprises a means for coupling to a syringe body, the device being configured such that the needle and the syringe are in line with the longitudinal axis.

7. The device of claim 1, wherein the valve in the open position is configured to allow passage of a guide wire, introducer sheath or central line through the valve and second conduit into the first conduit and out the needle.

8. The device of claim 7, wherein the valve further comprises a partially closed position configured to maintain a seal around the guide wire, introducer sheath or central line.

9. A method of accessing a vasculature of a patient with a device, the device including a body portion having a needle at a first end, a syringe at a second end, a first conduit extending along a longitudinal axis between the first end and the second end, a second conduit oblique to the first conduit and in communication with the first conduit, and a valve, the method comprising:
inserting the needle into the vasculature of the patient;
opening the valve, the valve being in communication with the second conduit and configured to selectively seal an end of the second conduit, the valve being operatable between a closed position and an open position, wherein the open position is configured to provide access to the second conduit; and
introducing an object through the valve and second conduit into the first conduit and out the needle, wherein introducing the object is performed with the syringe still coupled to the second end.

10. The method of claim 9, wherein opening the valve comprises pinching the valve to move it from the closed position to the open position.

11. The method of claim 9, wherein the valve is operatable between the closed position and the open position in a direction that is transverse to the longitudinal axis of the device.

12. The method of claim 9, wherein the valve is operatable between the closed position and the open position in a direction that is parallel to the longitudinal axis of the device.

13. The method of claim 9, wherein the valve further comprises a partially closed position configured to maintain a seal around the object, wherein the object may comprise a guide wire, introducer sheath or central line.

14. A method, comprising:
causing a vascular access device to be manufactured and made available to a user, the vascular access device comprising:
a first conduit extending along a longitudinal axis between a first end and a second end, wherein the first end is configured for coupling to a needle and the second end is configured for coupling to a syringe;
a second conduit, oblique to the first conduit and in communication with the first conduit; and
a valve in communication with the second conduit and configured to selectively seal an end of the second conduit, the valve being operatable between a closed position and an open position, wherein the open position is configured to provide access to the second conduit such that an object may be introduced through the valve and second conduit into the first conduit and out the needle;
providing instructions to the user, the instructions recorded on a tangible medium and comprising:
inserting the needle into the vasculature of the patient;
opening the valve so as to provide access to the second conduit; and
introducing an object through the valve and second conduit into the first conduit and out the needle, wherein introducing the object is performed with the syringe still coupled to the second end.

15. The method of claim 14, wherein opening the valve comprises pinching the valve to move it from the closed position to the open position.

16. The method of claim 14, wherein the valve is operatable between the closed position and the open position in a direction that is transverse to the longitudinal axis of the device.

17. The method of claim 14, wherein the valve is operatable between the closed position and the open position in a direction that is parallel to the longitudinal axis of the device.

18. The method of claim 14, wherein the valve further comprises a partially closed position configured to maintain a seal around the object, wherein the object may comprise a guide wire, introducer sheath or central line.
